(19) United States
(12) Patent Application Publication
(10) Pub. No.: US 2025/0037275 A1
(43) Pub. Date: Jan. 30, 2025

(54) ANEURYSM MODELING AND RISK PREDICTION

(71) Applicant: University of Pittsburgh - of the Commonwealth System of Higher Education, Pittsburgh, PA (US)

(72) Inventors: Timothy K. Chung, Pittsburgh, PA (US); David A. Vorp, Pittsburgh, PA (US)

(21) Appl. No.: 18/716,079

(22) PCT Filed: Dec. 2, 2022
(86) PCT No.: PCT/US2022/051718

§ 371 Date: Jun. 3, 2024

Related U.S. Application Data
(60) Provisional application No. 63/285,571, filed on Dec. 3, 2021.

(51) Int. Cl. G06T 7/00; G16H 50/30

(52) U.S. Cl. CPC G06T 7/0012; G16H 50/30; G06T 2207/10028; G06T 2207/10081; G06T 2207/20084

(57) ABSTRACT

Methods, systems, and apparatus, including computer programs encoded on computer storage media, for modeling and risk assessments of abdominal aortic aneurysms. A computing system generates from a set of medical images a three-dimensional (3D) model of the aneurysm, the 3D model defining an outer wall of the aneurysm, an intraluminal thrombus (ILT) region of the aneurysm, and a luminal region of the aneurysm. The system can analyze the 3D model to determine respective values for at least one morphological feature of the aneurysm and at least one biomechanical feature of the aneurysm. Using the respective values for the at least one morphological feature of the aneurysm and the at least one biomechanical feature of the aneurysm, the system can generate a prediction of one or more outcomes related to the aneurysm.

### CROSS-REFERENCE TO RELATED APPLICATIONS

This application claims the benefit of priority to U.S. Patent Application Ser. No. 63/285,571, filed Dec. 3, 2021, the entire contents of which are incorporated by reference in their entirety.

### STATEMENT REGARDING FEDERALLY SPONSORED RESEARCH

This invention was made with government support under AG037120 awarded by the National Institutes of Health. The government has certain rights in the invention.

### BACKGROUND

### 1. Technical Field

This specification describes computational techniques for analyzing aneurysms and similar structures, including techniques for modeling aneurysms and predicting the likelihood of one or more outcomes for the aneurysms (e.g., a predicted risk of the aneurysm rupturing within a period of time).

### 2. Background

Abdominal aortic aneurysm (AAA) is a leading cause of death in westernized countries and affects an aging population. The adult abdominal aorta is typically 2 centimeters (cm) in diameter, and is defined as aneurysmal when the diameter grows to exceed 3 cm. If left untreated, AAA may continue to grow and eventually rupture, with resulting morbidity and mortality rates exceeding 85%. Cerebral aneurysm is also a leading cause of death globally, and affect a wide range of patient demographics based on age, sex, and locale within the cerebrovasculature.

Currently, vascular surgeons use a maximum diameter criterion, typically set at 5.5 centimeters for adults, for elective endovascular aneurysm repair or open surgery. When aneurysm exceed the maximum diameter criteria, vascular surgeons will perform elective endovascular repair (EVAR) surgery. Aneurysms that fall within the 3 cm to 5.5 cm range, typically referred to as “small aneurysms,” are relegated to monitoring-despite an estimated 13-23.4% of small aneurysms nonetheless leading to rupture. For example, FIG. 1 depicts an aneurysm that has developed in an aorta that has caused the aorta's outer diameter to exceed 3 cm. According to traditional clinical practice, the aneurysm is measured and if the outer diameter exceeds 5.5 cm then the patient may be deemed eligible for elective repair surgery. If the diameter does not exceed the 5.5 cm threshold, then the patient is typically not recommended or deemed eligible for surgical repair. The physician may instead recommend that the patient undergo periodic surveillance to monitor how the size of the aneurysm changes over time (e.g., every 6 months).

A host of patient factors contribute to the prognosis of an aneurysm over time, including gender (e.g., up to five times more prevalent in men than women), age, dyslipidemia, smoking, hypertension, family history and obesity. Although there are patient factors (mentioned previously) for small aneurysms, most surgeons will not perform elective surgery for small aneurysms due to the unknown risks of elective or emergent repair. All clinically sized aortic aneurysms (greater than the maximum diameter criterion) are typically recommended to be repaired unless the patient is not eligible. It is also important to note that some aneurysms may outlive their condition.

The maximum diameter criterion has been applied for clinical decision making with respect to other types of aneurysms as well. For example, cerebral aneurysm (CA) is a degenerative dilation of arteries associated with a localized weakness in the vessel wall within the brain. Without treatment, CAs can rupture, an often-fatal cerebrovascular event and among the leading causes of death in the United States. Upon discovery of CA, a clinician evaluates the CA's clinical status to assess its risk of rupture against the risk of interventional repair via surgical clipping, coil embolism, or by placing a flow diverting device. Current clinical guidelines suggest that the risk of rupture outweighs the risk of intervention when the maximum diameter of CA exceeds 7.0 mm. However, up to 35% of smaller-sized CAs have nonetheless been reported to rupture despite current clinical guidelines pointing toward no intervention.

### SUMMARY

This specification describes systems, methods, devices, and techniques for modeling abdominal aortic aneurysms, and predicting one or more outcomes related to the aneurysm in a patient. The risk assessments described in this specification can advantageously account for a greater range of risk factors than conventional assessments that rely either exclusively or primarily on the maximum diameter of an aneurysm.

For example, this specification discloses machine-learning techniques for training predictive models that can process not only clinical data for a patient, but also morphological and biomechanical features of the patient's aneurysm, e.g., features that may be derived from computed tomography (CT) scans or other high-quality 3D medical imaging. Through analysis of a broader range of risk factors, the risk assessments disclosed herein can produce more accurate predictions than those offered by conventional approaches (especially those that observe only a limited amount of information such as size or diameter of the aneurysm). Thus, higher-risk aneurysms can be more readily identified even when they are relatively small (e.g., less than 5 cm), and the risk associated with larger aneurysms (e.g., more than 5 cm) can be further stratified based on additional risk factors. The improved AAA risk predictions described in this specification can thus aid physicians and patients alike in assessing whether less frequent surveillance of an aneurysm, more frequent surveillance, or endovascular repair surgery is warranted. For example, a clinician may initially prescribe less frequent surveillance of a small aneurysm if the risk prediction models indicate that rupture is unlikely to occur for a long period of time (e.g., more than 5+ years in the future) or the likelihood of rupture or further clinical intervention being required at all is relatively low. A clinician may prescribe more frequent surveillance, on the other hand, if the prediction models indicate a higher likelihood of rupture or additional clinical intervention being required within a shorter period of time. Moreover, the disclosed techniques can remove barriers to higher-level analysis of medical images of a patient's aneurysm by automating a software pipeline to efficiently segment the images, build a computational 3D model, extract key morphological indices from 3D models, and perform biomechanical analysis through finite element analysis, computational fluid dynamics, or other simulation methods (fluid structure interaction) to extract key biomechanical indices (feature values) of the aneurysm.

In general, one innovative aspect of the subject matter described in this specification can be embodied in computer-implemented methods that include the actions of obtaining, by a system of one or more computers, a medical image set of a person, the medical image set comprising one or more images that each depict at least a portion of an abdominal aortic aneurysm. The system can generate, using the medical image set, a three-dimensional (3D) model of the aneurysm, the 3D model defining an outer wall of the aneurysm, an intraluminal thrombus (ILT) region of the aneurysm if present, a coil thrombus mass of a cerebral aneurysm if intervention has occurred, and a luminal region of the aneurysm. The system can analyze the 3D model of the aneurysm to determine respective values for at least one morphological feature of the aneurysm and at least one biomechanical feature of the aneurysm. Using the respective values for the at least one morphological feature of the aneurysm and the at least one biomechanical feature of the aneurysm, the system can generate a prediction of one or more outcomes related to the aneurysm.

These and other aspects further include one or more of the following features.

Analyzing the 3D model of the aneurysm can include calculating stresses in at least one of the outer wall, ILT region (if present), or luminal region of the aneurysm under one or more loading conditions. The one or more loading conditions can correspond to pressures that are estimated to result from blood flow through the luminal region.

Analyzing the 3D model of the aneurysm can include using the 3D model to perform a finite element analysis of the aneurysm under one or more loading conditions.

The system can obtain clinical data for the person, the clinical data indicating clinical attributes of the person apart from morphological or biomechanical features of the aneurysm. Generating the prediction of the one or more outcomes related to the aneurysm can include processing the clinical data along with the respective values for the at least one morphological feature and the at least one biomechanical feature of the aneurysm.

Using the respective values for the at least one morphological feature of the aneurysm and the at least one biomechanical feature of the aneurysm to predict the one or more outcomes related to the aneurysm can include evaluating a predictive model using the respective values as at least a subset of inputs to the model. The predictive model can include a decision tree ensemble. The decision tree ensemble can be structured to boost lower level decision trees in the ensemble. Other types of predictive models may also be employed, including artificial neural networks (e.g., feedforward, convolutional, and/or recurrent neural networks), support vector machines, regression models, or a combination of such models.

The system can analyze at least a portion of the medical image set to predict the respective values of the at least one morphological feature of the aneurysm or the at least one biomechanical feature of the aneurysm.

At least one biomechanical feature of the aneurysm can include average wall stress or tension at one or more locations of the aneurysm, peak wall stress or tension at one or more locations of the aneurysm, and a range of wall stress percentiles of the aneurysm.

The at least one morphological feature of the aneurysm can include tortuosity of the aneurysm, characteristics of the intraluminal thrombus (ILT) region such as a maximum, minimum, and/or average thickness of the ILT region, a volume of the aneurysm, a maximum, minimum, and/or average diameter of the outer wall and/or lumen of the aneurysm, an outer wall and/or luminal wall surface area, and/or others facets of the aneurysm's geometry.

The 3D model of the aneurysm can include a computational model adapted for use in a finite element analysis of the aneurysm. Generating the 3D model can include generating a point cloud of the aneurysm using image information from the medical image set, generating an initial 3D mesh of the aneurysm from the point cloud, generating a refined 3D mesh of the aneurysm, including isolating the ILT region of the aneurysm in the mesh based on a Boolean difference operation, converting the refined 3D mesh to a polysurface 3D model of the aneurysm, and generating the computation model of the aneurysm from the refined 3D mesh, including defining boundary conditions and material properties for each of the outer wall, the ILT region, and the luminal region of the aneurysm.

The medical image set can include a set of images obtained from computed tomography (CT) scans of an abdominal region of the person.

The system can segment images in the medical image set to extract a portion of the image that depicts the diseased artery (e.g., the aneurysm).

Predicting the one or more outcomes related to the aneurysm can include predicting a likelihood of aortic rupture, a likelihood of clinical intervention, and/or a likelihood of clinical re-intervention (e.g., for cerebral aneurysms that have experienced rebleeding). Predicting the one or more outcomes can further include predicting a time until one or more outcomes occur, e.g., an expected time until the aneurysm would rupture.

Predicting the one or more outcomes related to the aneurysm can include classifying the aneurysm into one of a plurality of risk categories, wherein the plurality of risk categories comprises a low risk category, a moderate risk category, and a high risk category, each risk category associated with a different clinical treatment or surveillance option. Additional striation of risk outcomes can also include temporal factors, not limited to time-to-intervention or rupture.

In general, another innovative aspect of the subject matter described in this specification can be embodied in computer-implemented methods that include the actions of obtaining model inputs that indicate values of at least one clinical feature of a person, at least one morphological feature of an aortic aneurysm of the person, and at least one biomechanical feature of the aortic aneurysm of the person; and processing the model inputs with a predictive model to predict one or more outcomes related to the aneurysm.

These and other aspects further include one or more of the following features.

Obtaining the model inputs can include using a 3D model to simulate blood flow through a lumen of the aorta in a region of the aortic aneurysm, and evaluating results of the simulation.

The actions can further include processing a first portion of the model inputs that indicate the at least one value of the at least one clinical feature of the person with a first sub-model to generate a first preliminary prediction for the one or more outcomes of the aneurysm; processing a second portion of the model inputs that indicate the at least one value of the at least one morphological feature of the aortic aneurysm with a second sub-model to generate a second preliminary prediction for the one or more outcomes of the aneurysm; processing a third portion of the model inputs that indicate the at least one value of the at least one biomechanical feature of the aortic aneurysm with a third sub-model to generate a third preliminary prediction for the one or more outcomes of the aneurysm; and generating a final prediction for the one or more outcomes of the aneurysm based on a combination of the first, second, and third preliminary predictions.

Other embodiments of these aspects include corresponding computer systems, apparatus, and computer programs recorded on one or more computer storage devices, each configured to perform the actions of the methods. A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

The details of one or more implementations of the subject matter described in this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 illustrates the conventional application of a maximum diameter criterion for assessing whether to recommend elective endovascular surgery to repair an abdominal aortic aneurysm (AAA).

FIG. 2 depicts an example environment of a computing system for modeling an aneurysm from image data and generating an AAA risk prediction.

FIG. 3 is a flowchart of an example process for modeling an aneurysm and generating an AAA risk prediction.

FIG. 4 is a flowchart of an example process for generating a computational three-dimensional (3D) model of an abdominal aortic aneurysm.

FIG. 5 is a diagram of components of an exemplary AAA risk prediction model.

FIG. 6 is a diagram of an example process for training an AAA risk prediction model.

FIG. 7 depicts an example use of machine-learning techniques for segmenting and extracting depictions of an abdominal aortic aneurysm from a medical image set.

FIG. 8 depicts a 3D model of an abdominal aortic aneurysm with shading to indicate gradients in wall stresses determined through finite element analysis.

FIG. 9 illustrates certain example morphological indicates of an abdominal aortic aneurysm.

FIG. 10 is a CT image of a patient's abdominal region with labels indicating structures of an abdominal aortic aneurysm.

FIG. 11 depicts a refined 3D mesh of an abdominal aortic aneurysm.

FIG. 12A is an image of a cerebral aneurysm (CA), and FIG. 12B depicts a 3D reconstruction of the CA.

FIGS. 13A-C show the results of finite element analysis performed on a 3D computational model of the CA from FIGS. 12A-12V to determine various localized biomechanical indices of the CA.

FIG. 14 is a block diagram of an example computer system that can be programmed and configured to carry out any of the computational techniques disclosed in this specification.

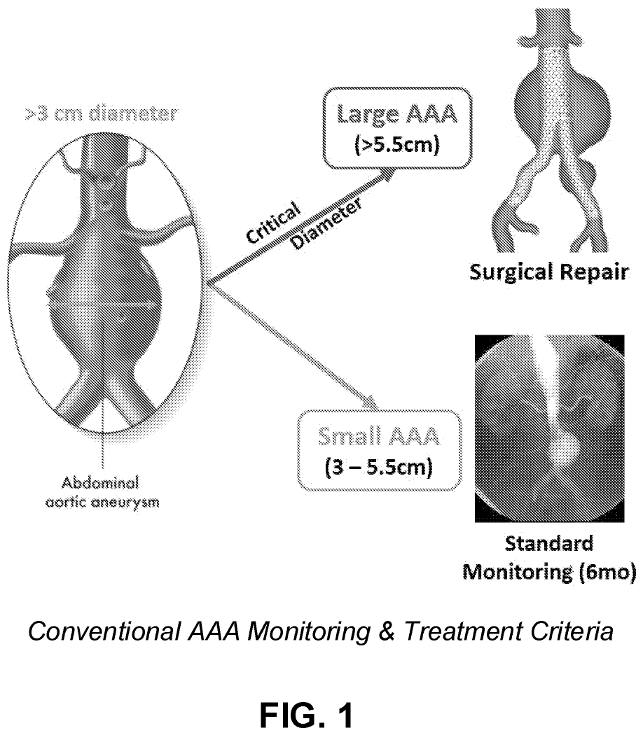

Like reference numbers and designations in the various drawings indicate like elements.

### DETAILED DESCRIPTION

This specification describes systems, methods, devices, and techniques for analyzing abdominal aortic aneurysms (AAA) and similar structures, and more particularly describes techniques for modeling aneurysms and predicting a risk of aortic rupture or other outcome related to an aneurysm in a patient.

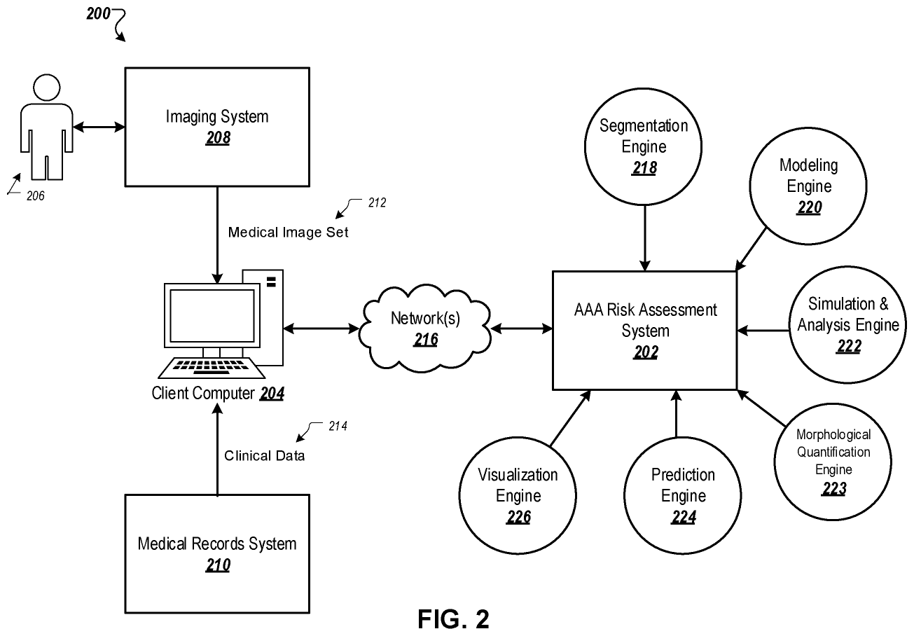

FIG. 2 depicts an example environment 200 of a computing system for modeling an aneurysm from image data and generating an AAA risk prediction. In general, the environment 200 encompasses one or more computers and associated equipment (e.g., imaging system 208) in one or more locations, and each of the individual systems 202, 204, 208, 210, 218, 220, 222, 223, 224, 226 can also be implemented on one or more computers in one or more locations. In some contexts, environment 200 provides for interaction between a local client computer 204 and a remote service (e.g., cloud-based service) executed on the AAA risk assessment system 202 in coordination with engines 218-226. For example, local entities such as physicians, hospitals, or clinics may subscribe or pay per-use fees to a provider of the AAA risk assessment service. The client computer 204 can send requests to the AAA risk assessment system 202 via one or more networks 216 (e.g., the Internet, local area networks (LANs), wireless area networks (WANs)), and the AAA risk assessment system 202, upon receiving all necessary inputs and data for analysis, may return reports to the client computer 204 containing AAA risk prediction(s) and/or modeling results to the client computer 204. A physician may review the reports to inform recommendations and treatment plans in relation to a patient's AAA.

The on-site environment can include an imaging system 208, client computer 204, and medical records system 210. Imaging system 208 is configured to obtain a set of medical images of a patient 206, and in particular, can be adapted to capture images that depict internal organs of the patient 206 including the aorta. Imaging system 208 can emit electromagnetic radiation toward an abdominal region of the patient 206 and record signal(s) detected in response to the radiation (e.g., reflected radiation) to produce an image of the targeted area. In some implementations, imaging system 208 is a computed tomography (CT) system that uses specialized X-ray equipment to produce a series of cross-sectional images of the patient's abdominal region. Each cross-sectional image represents a slice of the targeted region, for example. In other implementations, imaging system 208 is a magnetic resonance imaging (MRI) system in which the patient 206 is positioned in a magnetic field and the system directs radio waves toward the targeted region of the patient's body to obtain images of internal structures of the body. In general, imaging system 208 can include imaging equipment capable of acquiring any suitable form of medical images of a patient's anatomy that depict a targeted aneurysm with sufficient detail to permit reliable modeling, feature extraction, and measurements of the aneurysm. Different imaging modalities may be employed in some cases to image different types of aneurysms. For example, CT or MRI scans may be suitable for abdominal aortic aneurysms (AAA); 3D rotational angiography or digital subtraction angiography may be preferable in some cases for imaging cerebral aneurysms (CA).

Medical records system 210 is configured to maintain medical records for one or more patients, and can include one or more database(s) to store medical records in a structured format that facilitates indexing and accessing of specified records. Among other forms of medical records, system 210 can store clinical data 214 that describes information about a patient 206 apart from a medical image set 212 that is nonetheless clinically relevant to assessing the risk of an abdominal aortic aneurysm in the patient 206. For example, clinical data 214 can include indications of the patient's sex or gender, age, and whether the patient exhibits risk factors such as dyslipidemia, smoking, hypertension, obesity, family history of vascular issues, diabetes, body mass index, body surface area, height, weight, coronary heart disease, history of congenital heart failure, history of COPD, dialysis/kidney failure, sepsis, SIRS, septic shock, WBC count, hematocrit percentage, Marfan's, history of myocardial infarction (heart attack), angina, swollen legs, shortness of breath, bronchitis, other respiratory diseases, limited mobility pharmaceutical use, or co-morbidities. Clinical data can be queried in any suitable manner. For example, patient data in the medical records system 210 can be labeled with International Classification of Diseases (ICD) codes, and such data can be queried based on their corresponding ICD codes. The queried data can further be encoded (e.g., binary for presence/absence, or multiple integers to indicate a plurality of choices within a category).

Client computer 204 receives the set of medical images 212 acquired by imaging system 208 and clinical data 214 from medical records system 210, and generates a request for an AAA risk prediction for the patient 206. The request can be transmitted from client computer 204 to AAA risk assessment system 202 in one or more messages over networks 216, and can include all or a portion of medical image set 212 and clinical data 214 in format(s) suitable for processing by system 202.

In response to receiving a request from client computer 204, AAA risk assessment system 202 begins processing the request to generate an AAA risk prediction. The AAA risk prediction indicates a prediction for one or more possible outcomes related to the patient's aneurysm. Typically, the predicted outcomes include aortic rupture such that the AAA risk prediction would indicate a risk or likelihood of the patient's aorta rupturing due at least in part to the aneurysm. However, additional or alternative outcomes besides rupture can also be considered such as a likelihood that the aneurysm increases in size (e.g., diameter) by at least a specified amount or a likelihood that the aneurysm achieves at least a minimum size (e.g., 5.5 cm). Significantly, at the time of evaluation by the risk assessment system 202, the patient's aneurysm may still be considered “small” by traditional interpretations (e.g., <5.5 cm). The risk assessment system 202 may nonetheless incorporate additional clinical indices of the patient and morphological and biomechanical indices of the aneurysm to provide a richer, more holistic, and/or reliable prediction of the risk presented by a “small” aneurysm that would otherwise be available by a maximum diameter criterion alone. In some implementations, the AAA risk prediction includes a risk score that indicates a likelihood (e.g., probability) of the one or more possible outcomes related to the aneurysm being actualized. In some implementations, the AAA risk prediction includes a classification that indicates a relative stratification of the risk presented by the aneurysm. For example, the risk assessment system 202 may apply pre-defined thresholds to the risk score to classify a patient's risk as high, medium, or low. Each classification can correspond to a different clinical recommendation, e.g., a high risk prediction can correspond to a recommendation for elective endovascular repair of the aneurysm, a medium risk prediction can correspond to a recommendation for more frequent surveillance/monitoring of the aneurysm (e.g., schedule the patient for follow-up appointments every 3 months), and a low risk prediction can correspond to a recommendation for less frequent surveillance/monitoring of the aneurysm (e.g., schedule the patient for follow-up appointments every 6-12 months).

AAA risk assessment system 202 employs multiple processing engines 218, 220, 222, 223, 224, and 226 to process the request data and generate an AAA risk prediction. Although depicted in FIG. 2 as distinct modules, all or some of the engines 218, 220, 222, 223, 224, and 226 may be merged or further split into separate processing phases according to the demands of a particular application, and moreover all or some of the engines 218, 220, 222, 223, 224, and 226 can be implemented within or apart from system 202. In general, a processing “engine” in this specification refers to hardware, software, or both (including firmware) configured to perform one or more functions on one or more data processing apparatus. Collectively, the AAA risk assessment system 202 in cooperation with engines 218, 220, 222, 223, 224, and 226 provide a fully automated pipeline for analyzing a medical image set 212 and clinical data 214 to generate a computational 3D model of an abdominal aortic aneurysm, and to determines values for mechanical features of the aneurysm from the 3D model for use in generating an AAA risk prediction. Segmentation engine 218 processes all or some of the images in the medical image set 212 to isolate and extract the relevant portions of the images that depict the aorta in an aneurysmal region. Modeling engine 220 processes the segments of the medical image set 212 extracted by segmentation engine 218 to build the 3D model of the aneurysm.

With the 3D model constructed, simulation and analysis engine 222 performs a stress analysis and simulates blood flow through the aneurysm, thereby facilitating computation of stresses on the walls and other geometries of the aneurysm that could lead to rupture or other adverse outcomes. The simulation can involve finite element analysis, computational fluid dynamics simulations, analysis of fluid-structure interactions, or a combination of these and/or other analyses. System 202 uses results from the finite element analysis or other simulation results from engine 222 to extract respective values for one or more biomechanical features of the patient's aneurysm. Biomechanical features of the wall, lumen, and ILT can be taken under consideration. Biomechanical features can include average wall stress or tension at one or more locations of the aneurysm, peak wall stress or tension at one or more locations of the aneurysm, average Mises stress, average max Principal Stress, failure strength, failure tension, peak min wall tension, peak rupture potential index (RPI) tension, average max tension, average min tension, inter quartile range (IQR) Mises, IQR stress, IQR RPI, IQR RPI tension, IQR min principal stress, IQR max principal stress, IQR peak wall tension, IQR Mises stress, all aforementioned metrics based on any range of percentiles, and others.

Morphological quantification engine 223 analyzes the segmented medical images, computational 3D model, or both to measure one or more morphological features of the aneurysm. These morphological measurements, or morphological feature values, can include tortuosity of the aneurysm, characteristics of the intraluminal thrombus (ILT) region such as a maximum, minimum, and/or average thickness of the ILT region, a volume of the aneurysm, a maximum, minimum, and/or average diameter of the outer wall and/or lumen of the aneurysm, an outer wall and/or luminal wall surface area, diameter-height ratio, height ratio, diameter-diameter ratio, bulge location, asymmetry factor tortuosity, ILT volume, isoperimetric ratio, non-fusiform index, surface area of AAA sac, neck diameter, height of aneurysm sac, max principal curvature, min principal curvature, distal neck diameter, proximal neck diameter, maximum transverse diameter, lumen asymmetry, wall asymmetry, aspect ratio (height/diameter), length of AAA sac centerline, length of neck centerline, height of neck, distance between lumen centroid and centroid of maximum diameter cross section, area-averaged Gaussian curvature, mean curvature, and/or others facets of the aneurysm's geometry.

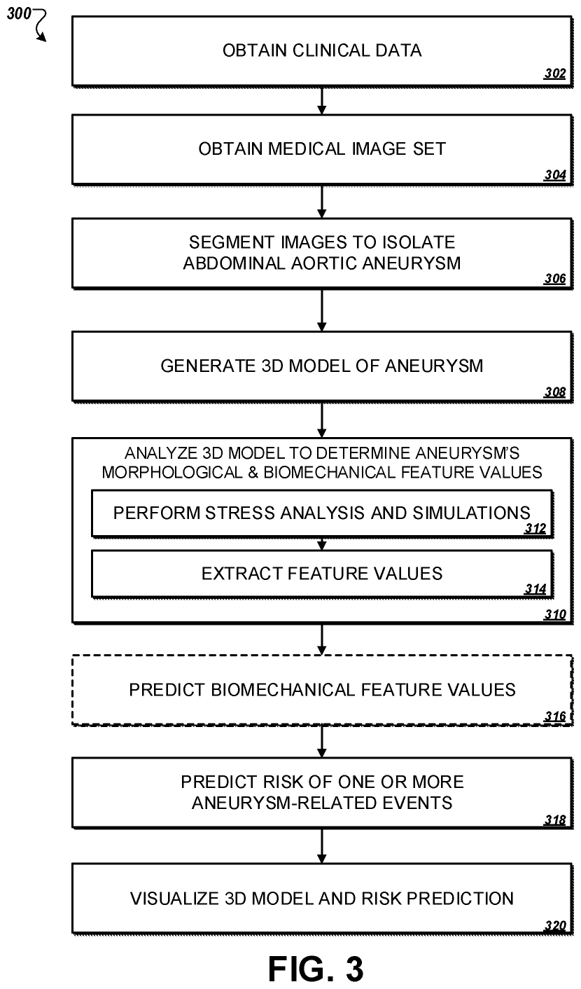

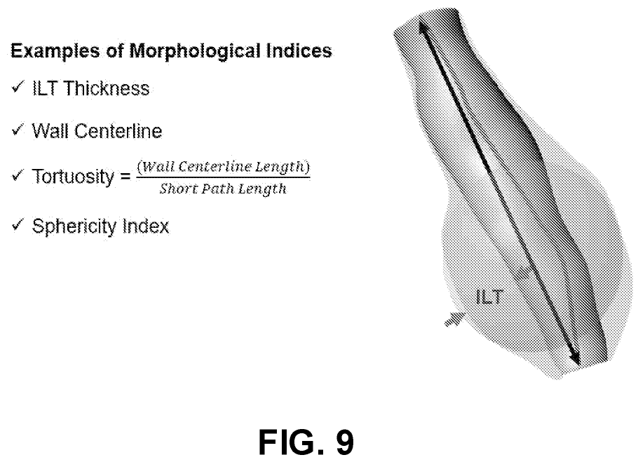

FIG. 9 illustrates dimensions of certain example morphological indices. System 202 also obtains clinical feature values for the patient 206 from the clinical data 214 submitted with the request, and provides the respective values for the clinical feature(s), biomechanical feature(s), and morphological feature(s) to prediction engine 224. Prediction engine 224 uses these feature values to evaluate one or more predictive models and generate an AAA risk prediction for the patient 206, which is returned as an independent value or in a report to the client computer 204 over networks 216. Additional details on operations performed by AAA risk assessment system 202 and engines 218, 220, 222, and 224 are described with respect to FIGS. 3-6.

FIG. 3 is a flowchart of an example process 300 for modeling an abdominal aortic aneurysm and generating an AAA risk prediction. Process 300 can be carried out by a system of one or more computers in one or more locations, such as the computing systems depicted in FIG. 2. At stage 302, the system obtains clinical data for the patient. A patient or physician may directly input clinical data to the system through suitable interfaces such a native software interface or a web-based portal. The system may also extract clinical data from existing data in a medical records system. Example predictive features described in the clinical data may include indications of the patient's sex or gender, age, and whether the patient exhibits risk factors such as dyslipidemia, smoking, hypertension, obesity, family history of vascular issues, pharmaceutical use, and/or co-morbidities.

At stage 304, the system obtains a set of medical images of a patient. The medical image set can include a stack of many (e.g., tens or hundreds) of images, each representing a slice of a targeted region of the patient's body such as the abdominal region. In some implementations, the images are obtained from CT scans of the patient. The original medical images can have very high resolution, each depicting an entire cross-section of the patient's abdomen that includes both the aorta and other internal structures and organs.

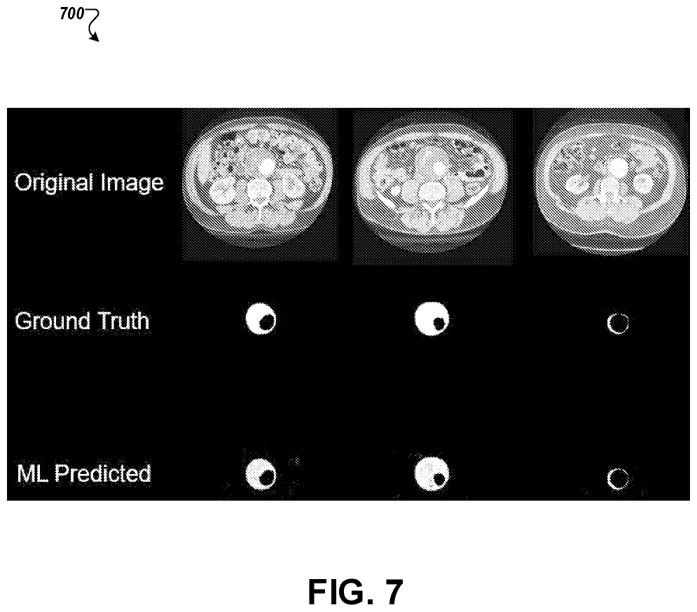

At stage 306, the system, e.g., segmentation engine 218, segments all or some of the medical images in stack to isolate and extract the relevant portion of each image where the aorta is located. If the patient has an aortic aneurysm, a slice of the aneurysm may be shown in the extracted portion of the image. Traditionally, image segmentation has been a tedious task performed manually by a trained clinician. But manual segmentation is expensive, time consuming, and often limited in the number of images that can be manually segmented within the available time and resources allocated to the task. To more efficiently segment a greater number of images in a shorter amount of time, the segmentation process can be automated through the use of machine-learning models that are trained to search for structures resembling an aneurysmal aorta in a medical image and to automatically isolate and extract the portion of the image showing the aorta. FIG. 7, for example, shows three images depicting the cross-section of a patient's abdomen obtained through CT imaging. A U-Net classifier can be trained on labeled images that identify the ground truth aortic structure in the image to later perform inference operations on new images and automatically segment the images with either no user intervention or minimal intervention. U-Nets are described, for example, in Olaf Ronneberger, Phillipp Fischer, and Thomas Brox's paper “U-Net: Convolutional Networks for Biomedical Image Segmentation,” available at https://arxiv.org/abs/1505.04597, which is incorporated by reference in its entirety into the contents of this disclosure.

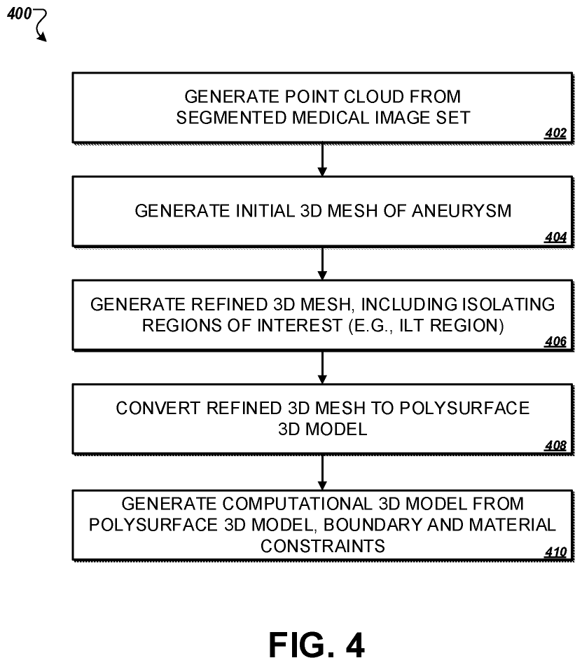

At stage 308, the system, e.g., modeling engine 220, processes the images of the aortic aneurysm extracted at stage 304 to generate a 3D model of the aneurysm (i.e., a 3D model of the aorta in a region that encompasses the aneurysm). Additional detail of the operations for constructing the 3D model is described further below with respect to FIG. 4.

At stage 310, the system, e.g., simulation and analysis engine 222, analyzes the 3D model of the aneurysm to determine values for one or more biomechanical and morphological features of the aneurysm. Example morphological features include tortuosity of the aneurysm, characteristics of the intraluminal thrombus (ILT) region such as a maximum, minimum, and/or average thickness of the ILT region, a volume of the aneurysm, a maximum, minimum, and/or average diameter of the outer wall and/or lumen of the aneurysm, an outer wall and/or luminal wall surface area, and/or others facets of the aneurysm's geometry. Example biomechanical features include average wall stress or tension at one or more locations of the aneurysm, peak wall stress or tension at one or more locations of the aneurysm, and others. Morphological indices can be obtained by measuring corresponding dimensions of the aneurysm in the 3D model. Biomechanical indices can be obtained by subjecting the 3D model to forces that simulate blood flow through the aneurysmal region of the aorta, and optionally additional forces that the aneurysm may experience in vitro. For example, a pressure of 120 mmHg corresponding to typical systolic blood pressures may be applied as a force directed radially outward from the lumen. The 3D model can be computationally defined with geometries, material properties, and material interactions that substantially mimic the actual geometries, material properties and interactions of the real tissue in the patient's aortic aneurysm. When the outward force from simulated blood flow is applied to the computational model, portions of the aneurysm may experience expansion or compression that deform the geometry of the aneurysm or otherwise result in stresses that could lead to rupture. In some implementations, the system performs simulation by finite element analysis (stage 312) and the results of the analysis are curated for use as predictive inputs to the AAA risk prediction model (stage 314). Other simulation techniques as known to a skilled artisan may also be suitable for use along with or in lieu of FEA including, e.g., mesh-free computational methods, computational fluid dynamics (CFD), and fluid-structure-interaction (FSI). Additionally, in some cases, multiple simulations can be performed to apply different types and/or levels of forces to the 3D model in each iteration (e.g., across a range of blood pressures). Pressures and forces can also be tailored to the biology of the individual patient, e.g., by applying higher simulated blood pressures for patients whose actual measured blood pressures are higher than an average or nominal value and applying lower simulated blood pressures for patients whose actual measured blood pressures are lower than the average or nominal value. When multiple simulations are performed, the system may curate results from one or more of the simulations for use as predictive inputs to the AAA risk prediction model.

At stage 316, the system optionally predicts feature values for one or more biomechanical features of the aneurysm based on analysis of one or more images in the segmented image stack. For example, the system may access one or more neural networks or other machine-learning models that have been trained to process inputs representative of one or more image(s) of the aneurysm to generate predictive outputs indicative of average or peak outer wall stress, luminal wall stress, aneurysm expansion under typical or high systolic pressures, and/or other biomechanical indices. The biomechanical feature predictive models can be trained using supervisory learning such backpropagation with gradient descent, using labeled training images of the aorta with target outputs for the biomechanical feature values. The target outputs can be computed through finite element analysis of a computational 3D model of the aneurysm. In some implementations, the system can predict biomechanical feature values for the aneurysm in lieu of constructing a computational 3D model of the aneurysm to more efficiently determine biomechanical feature inputs to the AAA risk predictive model. In some implementations, the system can use both predicted biomechanical feature values and simulated biomechanical feature values obtained using a computational 3D model as inputs to the AAA risk predictive model. In some implementations, the system foregoes prediction of biomechanical feature values and relies exclusively on simulation results (e.g., finite element analysis) from a 3D model to obtain the biomechanical indices of an aneurysm. Additional techniques that can be applied to predict biomechanical indices are described in U.S. Patent Application Ser. No. 62/915,565, filed Oct. 15, 2019, the entire contents of which are incorporated by reference in their entirety into the disclosure of this specification. Techniques for predicting wall stress and other biomechanical indices of an aneurysm are further described in PCT/US2020/055511, filed Oct. 14, 2020 and published Apr. 22, 2021 as WO2021/076575, the entire contents of which are incorporated by reference in their entirety into the disclosure of this specification.

At stage 318, the system, e.g., prediction engine 224, generates an AAA risk prediction for the patient. The AAA risk prediction indicates a prediction for one or more possible outcomes related to the patient's aneurysm. In some implementations, the AAA risk prediction includes a risk score that indicates a likelihood (e.g., probability) of the one or more possible outcomes related to the aneurysm being actualized. In some implementations, the AAA risk prediction includes a classification that indicates a relative stratification of the risk presented by the aneurysm. For example, the risk assessment system 202 may apply pre-defined thresholds to the risk score to classify a patient's risk as high, medium, or low. Each classification can correspond to a different clinical recommendation, e.g., a high risk prediction can correspond to a recommendation for elective endovascular repair of the aneurysm, a medium risk prediction can correspond to a recommendation for more frequent surveillance/monitoring of the aneurysm (e.g., schedule the patient for follow-up appointments every 3 months), and a low risk prediction can correspond to a recommendation for less frequent surveillance/monitoring of the aneurysm (e.g., schedule the patient for follow-up appointments every 6-12 months).

At stage 320, the system, e.g., visualization engine 226, produces a visual representation of the 3D model of the aneurysm, the risk prediction, or both. In some implementations, the visualization is produced in a virtual, augmented, or mixed reality environment. For example, a 3D model of the aneurysm can be rendered at a first virtual location and information based on the risk prediction can be rendered at a second virtual location at least partially overlaid or to the side of the 3D model. The information rendered can include the risk prediction itself (e.g., a risk score, a risk category, a predicted time to event) and information derived from the risk prediction that may be useful in interpreting the risk prediction. For instance, the system may determine that the patient belongs to a particular demographic group based on age, sex, and/or other factors, and may provide information to the patient and clinician indicating how the patient's risk prediction statistically compares to other patients in the same or related demographic group. The 3D model may also be interactive, allowing the clinician and/or patient to rotate, pan, and zoom the aneurysm. The visualization engine 226 can further colorize and render labels over the 3D model to indicate localized wall stresses or other biomechanical indices of the aneurysm. Visualization engine 226 can produce both localized and remote views to allow the 3D model and risk predictions to be viewed both in the clinician's office and remotely for in-person or remote consultations.

FIG. 4 is a flowchart of an example process 400 performed by one or more computers, e.g., modeling engine 220, for generating a computational three-dimensional (3D) model of an abdominal aortic aneurysm. The modeling engine starts by generating a point cloud from the segmented medical images that depict slices of the aortic aneurysm (stage 402). Each slice can represent a portion of the aneurysm at a different point along a longitudinal axis of the aorta. The distance between each image in the set is a known variable that can be identified by the modeling engine from image metadata or elsewhere, and this information can be used to stack the images in alignment to reconstruct the aneurysm in a third dimension, i.e., the dimension normal to the individual images. The point cloud can then be created in three dimensions by selecting in each image in the stack a collection of points that define the boundaries and/or inner structures of the aneurysmal aorta. For example, image processing algorithms can be applied to identify edges corresponding to the outer wall of the aneurysm as well as the luminal wall in the interior of the aorta, and points along the peripheries of these walls can be selected for the point cloud. By selecting points from each image or slice in the stack, the point cloud can be rendered in three dimensions.

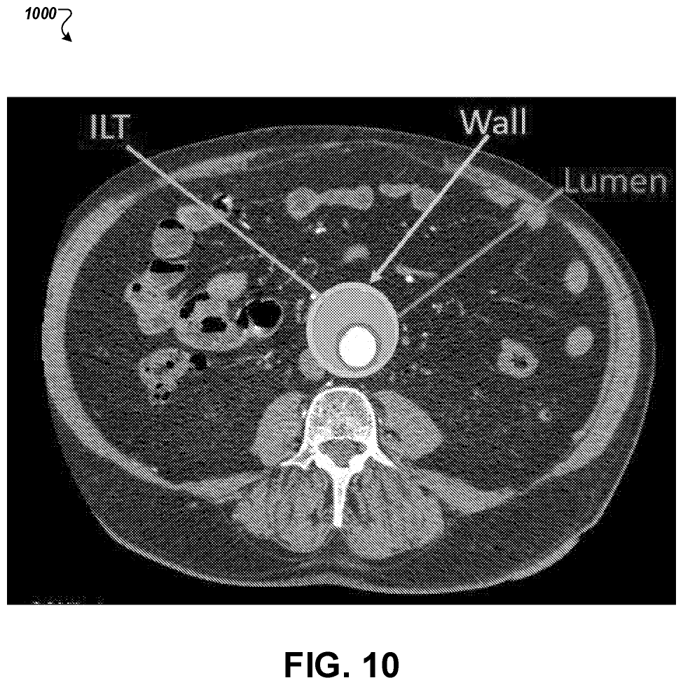

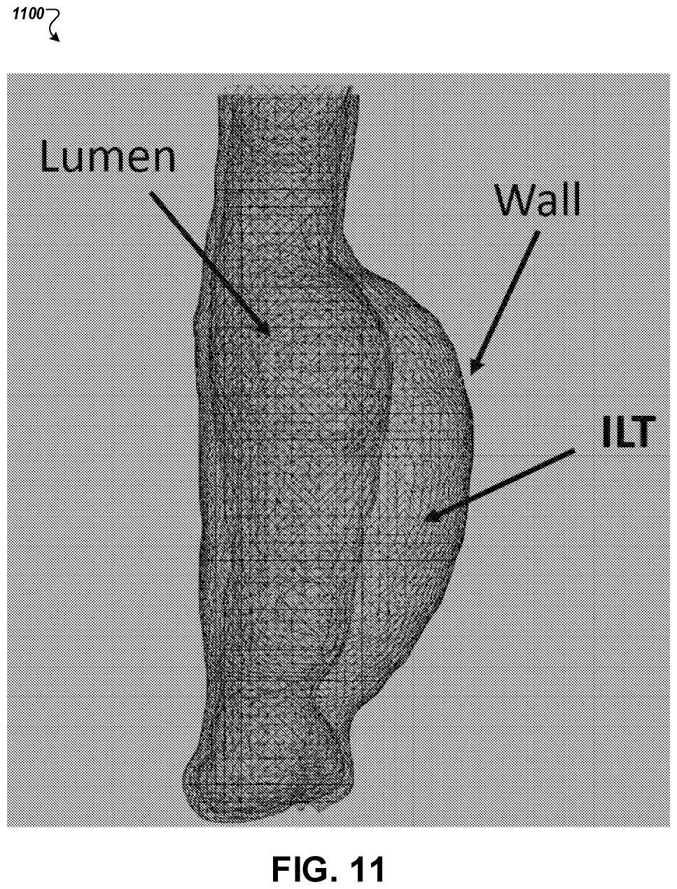

The modeling engine generates an initial 3D mesh of the aneurysm (stage 404). The initial 3D mesh can be formed by connecting points from the point cloud with edges to their nearest neighbors, thereby forming triangles or other polygons along the surfaces and contours of the virtual aneurysm. After creation of the initial 3D mesh, the system generates a refined mesh to isolate the intraluminal thrombus (ILT) region of the aneurysm (stage 406). In this disclosure, the ILT region refers to a volume or space between the lumen and outer wall of the aneurysm, e.g., as shown in FIGS. 10-11. The ILT region generally corresponds to a thickened region of an AAA due to blood clots. Other types of aneurysms such as cerebral aneurysms (CA) and ascending thoracic aneurysms (ATA) may not have ILT regions, but may nonetheless have other regions that could be isolated in the 3D mesh (e.g., coil thrombus mass region in CA).

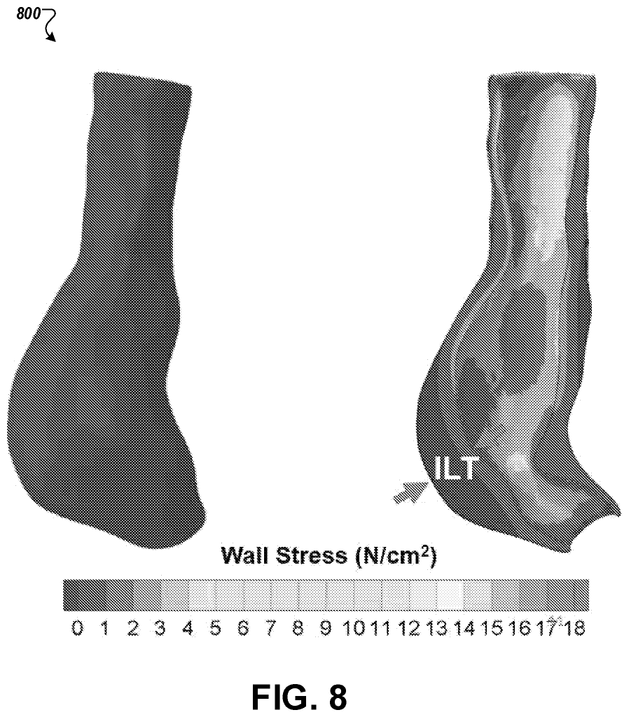

To isolate the ILT or coil thrombus mass region, the system in some implementations executes any combination of one or more Boolean operations to result in a subtraction of the luminal wall from the outer wall of the aneurysm. By way of illustration, FIG. 11 depicts an example refined 3D mesh that defines the luminal wall, outer wall, and ILT region in the intervening space between the luminal and outer walls. The refined 3D mesh is converted to a polysurface 3D model (stage 408). Unlike the initial or refined 3D meshes, the polysurface 3D model is mathematically defined in that the surfaces in the converted model are described by mathematical relationships with respect to each other rather than then mere spatial coordinates of the points in a mesh. From the polysurface model, the system (e.g., modeling engine 220) finally generates a computational 3D model of the aneurysm. The computational 3D model includes similar mathematically defined surfaces, but also defines boundaries and material properties for each of the outer wall, ILT region, and lumen (including the luminal wall) of the aneurysm. Material properties can be referenced to incorporate a wide-range of material models based on experimental testing uniaxially, biaxially, multi-axial inflation, biaxial inflation, compression, indentation, or radially that can be defined mathematically as a polynomial equation or other approaches to mimic the elastic properties of normal or diseased biological soft tissues. By way of example, FIG. 8 depicts one computational 3D model 800 of an aneurysm with shading to indicate gradients in wall stresses determined through finite element analysis.

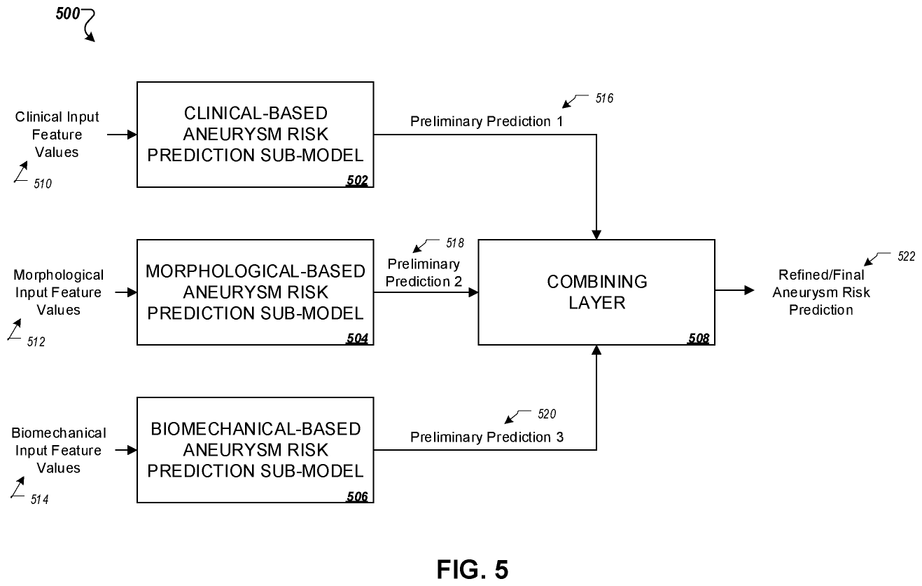

FIG. 5 is a diagram an exemplary AAA risk prediction model 500. Risk prediction model 500 is a machine-learned model with parameters that are learned, e.g., through supervised learning techniques. Due in part to the heterogeneous nature of inputs processed by the model 500, decision tree models such as random forests and ensemble tree models with boosting of lower-level decision trees have performed suitably well in empirical studies. However, other forms of the machine-learning models may also be employed, including artificial neural networks (e.g., feedforward, convolutional, and/or recurrent neural networks), support vector machines, or regression models.

In some implementations, prediction model 500 includes multiple components configured to generate AAA risk predictions in phases. In a first phase, a collection of sub-models 502, 504, and 506 generate preliminary predictions 516, 518, 520 based on different subsets of predictive inputs. For example, a first sub-model 502 is specially adapted to process clinical input feature values 510 to generate a first preliminary prediction 516. A second sub-model 504 is specially adapted to process morphological input feature values 512 to generate a second preliminary prediction 518. A third sub-model 506 is specially adapted to process biomechanical feature values 514 to generate a third preliminary prediction 520. The sub-models 502, 504, 506 may be individually or collectively trained, and sub-models 502, 504, 506 may have the same or different architectures from each other. Each of the preliminary predictions 516, 518, 520 indicates a preliminary assessment of the risk condition of an aortic aneurysm expressed as a risk score (e.g., a probability score relating to the probability of rupture or other outcome), a classification (e.g., stable, medium risk, high risk), or both. While the preliminary predictions 516, 518, 520 reflect predictions of the aneurysm's risk based on respective subsets of the predictive inputs, the final AAA risk prediction 522 is determined in a second stage by accounting for a combination of preliminary predictions 516, 518, 520. The combining layer 508 combines the preliminary predictions 516, 518, 520 using suitable weighting criteria. For instance, the first preliminary prediction 516 may be weighted lower than the other preliminary predictions 518, 520 if the accuracy of sub-model 502 is known to be lower than the respective accuracies of sub-models 504, 506. In some cases, apportioning risk prediction model 500 into sub-models corresponding to different subsets or categories of predictive features is beneficial to promote efficiency and flexibility in training and re-training the overall model 500. For example, sub-models that are individually trained can be more efficiently trained on additional training samples and may be adapted in various ways that do not necessarily impact or require re-training of other sub-models.

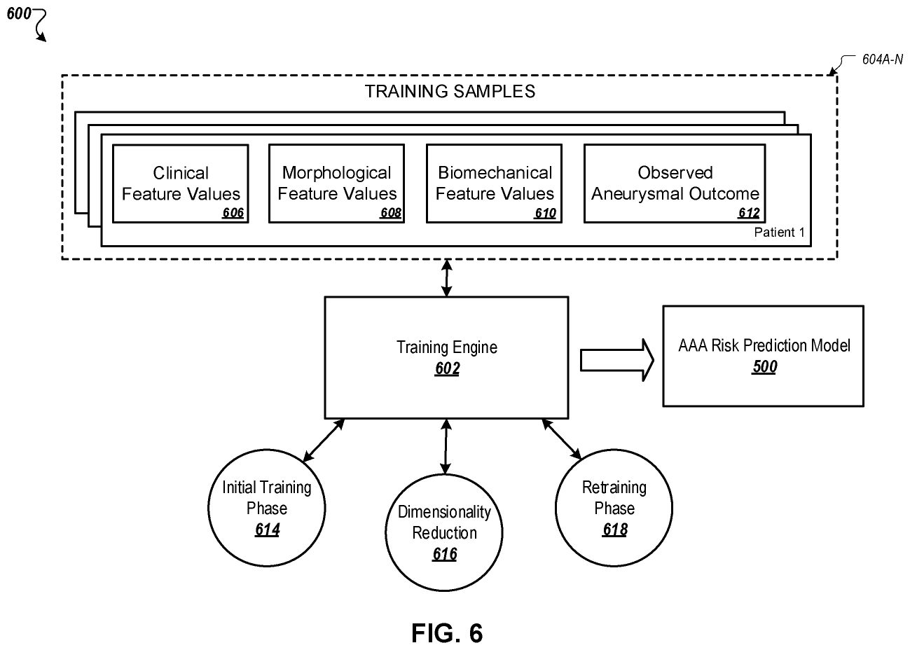

FIG. 6 is a diagram of an example process 600 for training an AAA risk prediction model 620. A training engine 602 obtains a collection of training samples 604A-N. Each training sample 604 corresponds to a respective patient, with some samples potentially corresponding to the same patient at different points in time and other samples corresponding to different patients. In general, each training sample 604 includes a set of clinical feature values 606, a set of morphological feature values 608, a set of biomechanical feature values 610, and an observed/target aneurysmal outcome 612. The clinical feature values 606 indicate non-imaged clinical data of the patient, the morphological feature values 608 indicate morphological indices of the patient's aneurysm, and the biomechanical feature values 610 indicate biomechanical indices of the patient's aneurysm (e.g., based on 3D modeling and finite element analysis of the aneurysm). The observed aneurysmal outcome 612 is the target classification and represents a final outcome of the aneurysm. For example, the feature values 606, 608, 610 may represent measurements from a patient's appointment while the aneurysm was still “small” (e.g., <5.5 cm). Based on continuing surveillance of the aneurysm, data sets may reveal that within a certain amount of time (e.g., 6 months, 1 year, 2, years, 5 years), the aneurysm either remained small (stable), the aneurysm ruptured, or the aneurysm grew large such that surgical repair was undertaken or recommended. The observed aneurysmal outcome 612 can indicate which of these outcomes resulted for the patient's aneurysm.

In some implementations, a temporal component may be added to process data longitudinally based on the number of serial imaging sessions a patient may undergo during the course of monitoring of an aneurysm. Generally, any clinical, biomechanical, and/or morphological feature used in training the models and making inference predictions, and whose value(s) are susceptible to changing over time, can have a corresponding derivative whose value indicates a rate of change of the feature over time. The derivatives can be used as predictive inputs to the AAA risk prediction model additionally or alternatively to the original, non-derivative features. First order derivatives indicating a rate of change of the feature's value over time, second order derivatives indicating accelerations of the feature's value over time, and/or higher-order derivatives can all be employed in training and making inferences with the AAA risk prediction model. For example, the peak wall stress and rate of change of peak wall stress of an aneurysm can both be provided as inputs to the risk prediction model. Likewise, changes in features of the intraluminal thrombus over time can be provided as inputs to the model. Rates of change for each quantity can be used to train the AAA risk prediction model (or another classification or regression model) for both patient outcome and estimated time-to-event (clinical intervention, rupture, or death).

In some implementations, the observed/target outcome 612 for each training sample can include a time to event indicator that indicates a length of time elapsed between (i) a time when measurements of the aneurysm or patient were obtained for the predictive inputs 606, 608, and 610 and (ii) a time when the observed/target outcome 612 is deemed to have been achieved. For example, a training sample may include predictive inputs 606, 608, and 610 that describe clinical, biomechanical, and morphological indices of an aneurysm 3.5 years before it ruptures. The observed/target outcome 612 can thus be defined with one or more values that indicate an expected 3.5 years until rupture of the aneurysm given the values of the predictive inputs 606, 608 and 610. With such techniques, a trained risk prediction model can be used during inference to make predictions that indicate not only the likelihood that an aneurysm will realize each possible target outcome defined by the classifier but also the expected time until the outcome is realized (e.g., the trained model could generate a prediction indicating that data for a newly presented aneurysm is expected to rupture within 3.5 years).

In some implementations, training engine 602 performs an initial training phase 614 to train an initial version of risk prediction model 500. The initial version of model 500 may be configured to generate predictions based on a relatively large number of predictive inputs for many clinical, morphological, and biomechanical features. The training engine 602 may then perform principal component analysis (PCA) or other techniques to identify the most salient features, e.g., those features that have the greatest predictive power and contribute the most to influencing accurate risk predictions in the model. The training engine 602 may then perform dimensionality reduction 616 by isolating the subset of most salient features from the remaining features in the original feature set, in a retraining phase 618, retrains the model 500 based only on the subset of most salient features (e.g., top n features). Reducing the number of dimensions or variables in risk prediction model 500 is significant to reduce the overall model size and to increase efficiency in evaluating the model. A smaller model 500 can be stored in less memory, requires fewer operations to evaluate, and reduces the burden of collecting, measuring, and computing predictive inputs (feature values) that contribute little to the ultimate risk prediction. In some implementations, the various sub-models of model 500 are trained jointly by generating a final prediction using the preliminary predictions from each sub-model, comparing the final prediction to the target prediction, and adjusting parameters in each sub-model based on any error between the predicted value and target value. In other implementations, the various sub-models are trained independently, e.g., by comparing the preliminary predictions to the target prediction and independently adjusting parameters in the sub-model based on the comparison.

Embodiments of the subject matter described in this specification can further be applied to model and assess the risk of rupture or other outcomes/events for aneurysms other than abdominal aortic aneurysms (AAA). The present techniques can be applied, for example, to analyze cerebral aneurysms (CA), ascending thoracic aortic aneurysms (ATAA), or both. In general, the approach to modeling and analyzing these additional types of aneurysms are similar to those described in this specification with respect to AAA. Similar medical imaging procedures can be performed to acquire internal images of the patient's anatomy in a region of the aneurysm; the images are segmented to isolate the diseased vessel; and the segmented images are processed to create a 3D model of the aneurysm. These operations can be performed by an automated software pipeline with minimal to no human intervention, and the pipeline can be further programmed to analyze the images and/or 3D model to determine values for various biomechanical and morphological features of the aneurysm. The values for biomechanical features can be obtained using any suitable approach, including through simulations, finite element analysis, machine-learning models (e.g., with neural network(s) or other known types of machine-learning models), or a combination of such techniques. Morphological feature values for the aneurysm can be obtained through image analysis of the aneurysm shown in one or more of the medical images, analysis of the 3D model, or both. Risk assessment model(s) on one or more computers can process the clinical, biomechanical, and/or morphological feature values to generate one or more predictions for the aneurysm, such as a score that indicates a likelihood of rupture or other clinical event, a classification that indicates a risk tier for the aneurysm with respect to a specified event (e.g., rupture), and/or a time to event indicator that indicates the predicted time to rupture of the aneurysm under study.

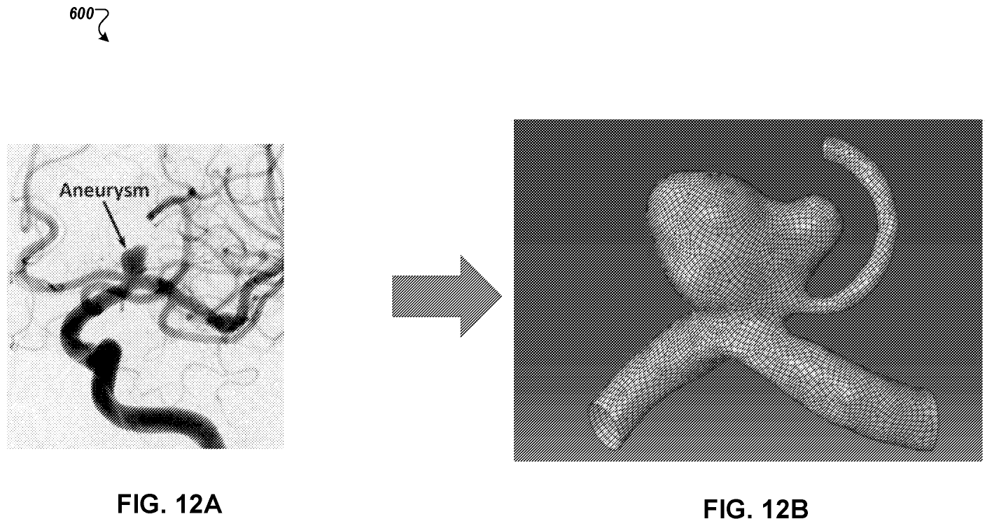

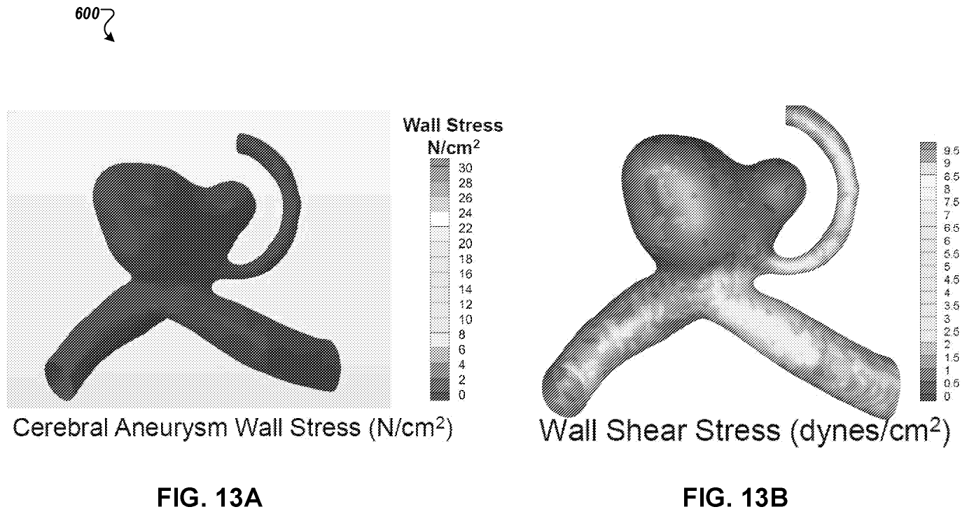

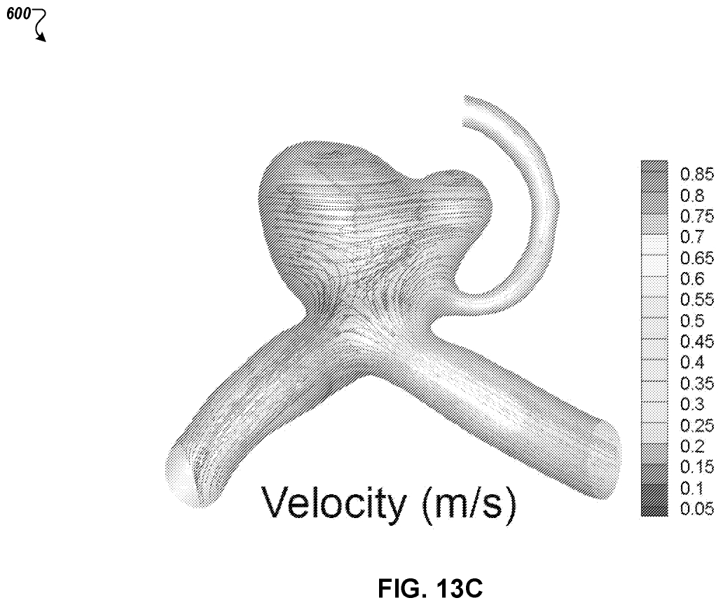

By way of example, FIG. 12A is an image of a cerebral aneurysm (CA) within the brain of a patient. FIG. 12B depicts a 3D model of the CA constructed from images like that shown in FIG. 12A, which can be used in simulations and other analyses to derive biomechanical and/or morphological features for the CA. FIGS. 13A-C depict results of FEA and CFD analysis to determine various biomechanical indices across and within the CA. Computational analysis simulating blood flow through the aneurysm may include extending the inlet region to allow flow to develop or become laminar. A pressure outlet boundary condition can be applied to fluid leaving the aneurysm. Pulsatile blood velocity can be used and rescaled accordingly to accommodate differences in cross-sectional area of the inlet, whereby affecting bulk flow through the diseased vasculature. Steady flow can also be defined as the peak velocity of the pulsatile blood waveform for simplified simulations. For fluid structure interaction simulations, a pressure pulse can be initiated to displace the aneurysm wall while allowing for blood fluid to be simulated.

At each stage of the pipeline, the software package executed in that stage can be selected or adjusted according to the type of aneurysm being analyzed in a particular procedure (e.g., AAA, CA, or ATAA). The differences in each stage can reflect intrinsic differences in the types of aneurysms at issue. In some implementations, for example, different predictive models can be trained for each type of aneurysm such that a first set of model(s) would be employed to generate risk predictions for AAA, a second set of model(s) would be employed to generate risk predictions for CA, a third set of model(s) would be employed to generate risk predictions for ATAA, and so on. While the respective model(s) for each type of aneurysm may be adapted to process similar categories of clinical, biomechanical, and morphological feature values to generate risk predictions, some or all (or none) of the particular features within each of the categories may vary across different types of aneurysms. For example, the clinical features used as predictive inputs to the models for CA and ATAA may differ in part from the clinical features used as predictive inputs to AAA models (e.g., features describing the different medications relevant to treating AAA, CA, and ATAA). The respective biomechanical and morphological features used as predictive inputs to the risk assessments models for AAA, CA, and ATAA may also different from each other in part or in or whole. Notably, for example, features pertaining to the intraluminal thrombus (ILT) can be particularly useful in predicting outcomes for AAA. ILT-related features are generally unavailable for CA or ATAA, however, since ILT regions do not naturally form in CA or ATAA. Features describing other structures or properties of CA and ATAA may instead be used as predictive inputs for these aneurysm types, such as coiled thrombus mass for CA. The coil thrombus mass for CA is created after an embolism procedure that may require reintervention if the mass compacts or aneurysm continues to grow (allowing for blood to re-enter the aneurysm sac). Additionally, the system may adjust the 3D models, simulation parameters, or both based on differences in the type of aneurysm at issue (e.g., AAA, CA, or ATAA), including adjusting material properties to account for differences in tissue between the different types of aneurysms. Similar types of biomechanical features values may be calculated for the relevant structures of the particular type of aneurysm as those described with respect to AAA, including wall stresses and shear stresses in the aneurysm under simulated in vivo conditions. Morphological features for CA can also include the aneurysm's measured diameter, diameter to height ratio, and sphericity.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory program carrier for execution by, or to control the operation of, data processing apparatus. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus. The computer storage medium can be a machine-readable storage device, a machine-readable storage substrate, a random or serial access memory device, or a combination of one or more of them.

The term “data processing apparatus” refers to data processing hardware and encompasses all kinds of apparatus, devices, and machines for processing data, including by way of example a programmable processor, a computer, or multiple processors or computers. The apparatus can also be or further include special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, a module, a software module, a script, or code, can be written in any form of programming language, including compiled or interpreted languages, or declarative or procedural languages, and it can be deployed in any form, including as a stand-alone program or as a module, component, subroutine, or other unit suitable for use in a computing environment. A computer program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application-specific integrated circuit).

Computers suitable for the execution of a computer program include, by way of example, general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks;
magneto-optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the user device, which acts as a client. Data generated at the user device, e.g., a result of the user interaction, can be received from the user device at the server.

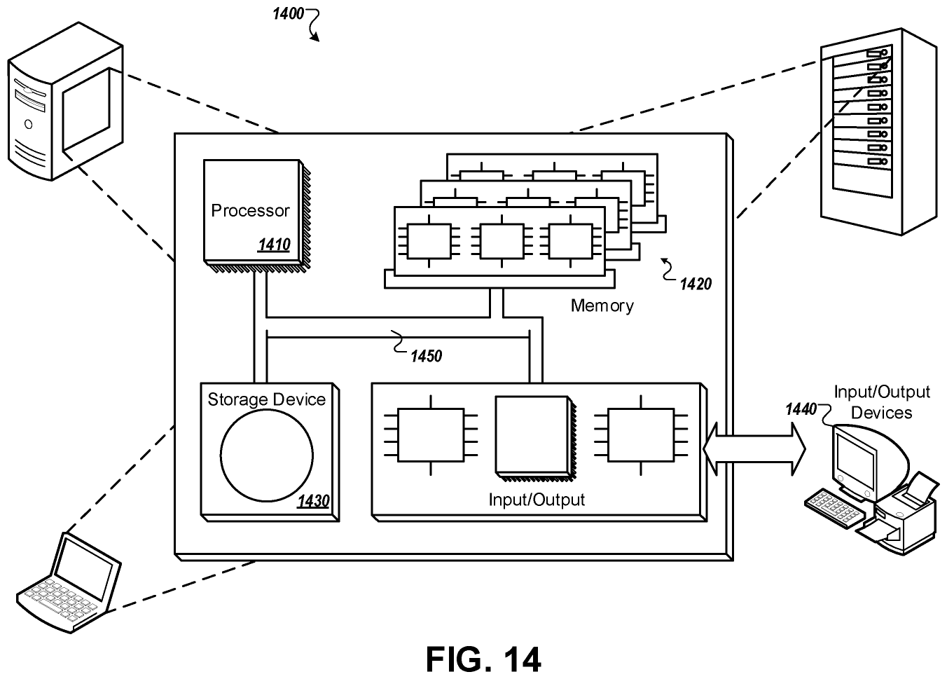

An example of one such type of computer is shown in FIG. 14, which shows a schematic diagram of a generic computer system 1400. The system can be used for the operations described in association with any of the computer-implemented methods described previously, according to one implementation. The system 1400 includes a processor 1410, a memory 1420, a storage device 1430, and an input/output device 1440. Each of the components 1410, 1420, 1430, and 1440 are interconnected using a system bus 1450. The processor 1410 is capable of processing instructions for execution within the system 1400. In one implementation, the processor 1410 is a single-threaded processor. In another implementation, the processor 1410 is a multi-threaded processor. The processor 1410 is capable of processing instructions stored in the memory 1420 or on the storage device 1430 to display graphical information for a user interface on the input/output device 1440.

The memory 1420 stores information within the system 1400. In one implementation, the memory 1420 is a computer-readable medium. In one implementation, the memory 1420 is a volatile memory unit. In another implementation, the memory 1420 is a non-volatile memory unit.

The storage device 1430 is capable of providing mass storage for the system 500. In one implementation, the storage device 1430 is a computer-readable medium. In various different implementations, the storage device 1430 may be a floppy disk device, a hard disk device, an optical disk device, or a tape device.

The input/output device 1440 provides input/output operations for the system 500. In one implementation, the input/output device 1440 includes a keyboard and/or pointing device. In another implementation, the input/output device 1440 includes a display unit for displaying graphical user interfaces.

While this specification contains many specific implementation details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method, comprising:
obtaining, by a system of one or more computers, a medical image set of a person, the medical image set comprising one or more images that each depict at least a portion of an abdominal aortic aneurysm;
generating, by the system and using the medical image set, a three-dimensional (3D) model of the aneurysm, the 3D model defining an outer wall of the aneurysm, an intraluminal thrombus (ILT) region of the aneurysm, and a luminal region of the aneurysm;
analyzing, by the system, the 3D model of the aneurysm to determine respective values for at least one morphological feature of the aneurysm and at least one biomechanical feature of the aneurysm; and
using the respective values for the at least one morphological feature of the aneurysm and the at least one biomechanical feature of the aneurysm to predict one or more outcomes related to the aneurysm.

2. The method of claim 1, wherein analyzing the 3D model of the aneurysm comprises calculating stresses in at least one of the outer wall, ILT region, or luminal region of the aneurysm under one or more loading conditions.

3. The method of claim 2, wherein the one or more loading conditions correspond to pressures that are estimated to result from blood flow through the luminal region.

4. The method of claim 1, wherein analyzing the 3D model of the aneurysm comprises using the 3D model to perform a finite element analysis of the aneurysm under one or more loading conditions.

5. The method of claim 1, comprising obtaining clinical data for the person, the clinical data indicating clinical attributes of the person apart from morphological or biomechanical features of the aneurysm, wherein predicting the one or more outcomes related to the aneurysm comprises processing the clinical data along with the respective values for the at least one morphological feature and the at least one biomechanical feature of the aneurysm.

6. The method of claim 1, wherein using the respective values for the at least one morphological feature of the aneurysm and the at least one biomechanical feature of the aneurysm to predict the one or more outcomes related to the aneurysm comprises evaluating a predictive model using the respective values as at least a subset of inputs to the model.

7. The method of claim 6, wherein the predictive model comprises a decision tree ensemble, and wherein the decision tree ensemble boosts lower level decision trees in the ensemble.

8. The method of claim 1, comprising analyzing at least a portion of the medical image set to predict the respective values of the at least one morphological feature of the aneurysm or the at least one biomechanical feature of the aneurysm.

9. The method of claim 1, wherein the at least one biomechanical feature of the aneurysm comprises an indication of wall stress or tension at one or more locations of the aneurysm.

10. The method of claim 1, wherein the at least one morphological feature of the aneurysm comprises a tortuosity of the aneurysm or a dimension of the ILT region.

11. The method of claim 1, wherein the 3D model of the aneurysm is a computational model adapted for use in a finite element analysis of the aneurysm, wherein generating the 3D model comprises:
generating a point cloud of the aneurysm using image information from the medical image set;
generating an initial 3D mesh of the aneurysm from the point cloud;
generating a refined 3D mesh of the aneurysm, including isolating the ILT region of the aneurysm in the mesh based on a Boolean difference operation;
converting the refined 3D mesh to a polysurface 3D model of the aneurysm; and
generating the computation model of the aneurysm from the refined 3D mesh, including defining boundary conditions and material properties for each of the outer wall, the ILT region, and the luminal region of the aneurysm.

12. The method of claim 1, wherein the medical image set comprises a set of images obtained from computed tomography (CT) scans of an abdominal region of the person.

13. The method of claim 1, comprising segmenting images in the medical image set to identify a portion of the image that depicts the aorta.

14. The method of claim 1, wherein predicting the one or more outcomes related to the aneurysm comprises predicting a likelihood of aortic rupture.

15. The method of claim 1, wherein predicting the one or more outcomes related to the aneurysm comprises classifying the aneurysm into one of a plurality of risk categories, wherein the plurality of risk categories comprises a low risk category, a moderate risk category, and a high risk category, each risk category associated with a different clinical treatment or surveillance option.

16. The method of claim 1, wherein predicting the one or more outcomes related to the aneurysm comprises predicting an amount of time until a particular outcome will occur.

17. The method of claim 16, wherein the particular outcome is a rupture of the aneurysm.

18. The method of claim 1, comprising determining a derivative of a biomechanical or a morphological feature of the aneurysm, wherein the one or more outcomes related to the aneurysm are predicted further based on the derivative of the biomechanical or the morphological feature of the aneurysm.

19. The method of claim 1, comprising processing images of the aneurysm with a neural network to generate a predicted wall stress of the aneurysm.

20. One or more non-transitory computer-readable media having instructions stored thereon that, when executed by one or more processors, cause performance of operations comprising:
obtaining, by a system of one or more computers, a medical image set of a person, the medical image set comprising one or more images that each depict at least a portion of an abdominal aortic aneurysm,
generating, by the system and using, the medical image set, a three-dimensional (3D) model of the aneurysm, the 3D model defining an outer wall of the aneurysm, an intraluminal thrombus (ILT) region of the aneurysm, and a luminal region of the aneurysm;
analyzing, by the system, the 3D model of the aneurysm to determine respective values for at least one morphological feature of the aneurysm and at least one biomechanical feature of the aneurysm; and
using the respective values for the at least one morphological feature of the aneurysm and the at least one biomechanical feature of the aneurysm to predict one or more outcomes related to the aneurysm.

21. A system comprising:
one or more computers; and
one or more computer-readable media having instructions stored thereon that, when executed by the one or more computers, cause performance of operations comprising:
obtaining, by a system of one or more computers, a medical image set of a person, the medical image set comprising one or more images that each depict at least a portion of an abdominal aortic aneurysm;
generating, by the system and using the medical image set, a three-dimensional (3D) model of the aneurysm, the 3D model defining an outer wall of the aneurysm, an intraluminal thrombus (ILT) region of the aneurysm, and a luminal region of the aneurysm;
analyzing, by the system, the 3D model of the aneurysm to determine respective values for at least one morphological feature of the aneurysm and at least one biomechanical feature of the aneurysm; and
using the respective values for the at least one morphological feature of the aneurysm and the at least one biomechanical feature of the aneurysm to predict one or more outcomes related to the aneurysm.

22-33. (canceled)
